(19) United States
(12) Patent Application Publication
(10) Pub. No.: US 2010/0258213 A1
(43) Pub. Date: Oct. 14, 2010

(54) Transfer device

(76) Inventors: Steven Raymond, Bromont (CA); Daniel MacDonald, Bromont (CA); Julie Trepanier, Sherbrooke (CA)

Correspondence Address: Eric Fincham, 316 Knowlton Road, Lac Brome, QC, J0E 1V0, CA

(21) Appl. No.: 12/456,101

(22) Filed: Jun. 10, 2009

(30) Foreign Application Priority Data
Apr. 9, 2009 (CA) 2,662,331

(51) Int. Cl. B65B 1/04; B65B 3/04; A61M 5/00

(52) U.S. Cl. 141/1; 141/329; 604/181

(57) ABSTRACT

The transfer device for transferring a fluid from a container having a body with an open first end and a piston within the body, the transfer device comprising an outer housing which is designed to receive a cartridge assembly which includes the container, the plunger rod aligned with the piston in the body, a spring position to drive the plunger rod in a direction towards the piston, a retractor to move the plunger rod from an activated position wherein the spring is extended to a non-activated position wherein the spring is compressed, and an actuator to permit the spring to be released to drive the plunger rod to move the piston within the container body. Typically, the device is used for transferring fluid from one container to a further container.

### FIELD OF THE INVENTION

The present invention relates to a portion of a transfer device and more particularly, relates to a transfer device suitable for transferring fluid contents from a first container to a second container.

### BACKGROUND OF THE INVENTION

In the pharmaceutical field, medicants can be packaged in a first type of container and need to be transferred to a second type of container. The reason is frequently that the second type of container is a specialized one which cannot easily be filled on known production lines. Rather than incurring the expense of building a new filling line which can involve substantial expenses for the design thereof, it can be preferable to transfer the contents of the standard package to the different package by an automated fluid control system. This is particularly the case when the transfer is done by the consumer or user of the product.

### SUMMARY OF THE INVENTION

It is an object of the present invention to provide an actuator for a transfer system for transferring a fluid from a first container to a second container.

It is a further object of the present invention to provide a transfer device for transferring a fluid from a first container to a second container wherein the device is substantially automated such that consistent results are achieved every time the device is used.

According to one aspect of the present invention there is provided a transfer device for transferring a fluid from a container having a body, an open first end, and a piston within the body, a septum at a second end thereof, the transfer device comprising an outer housing, the outer housing having a receiving area to hold and retain a cartridge assembly which includes the container, a plunger rod aligned with the piston, a spring positioned to drive the plunger rod in a direction towards the piston, a retractor to move the plunger rod from an activated position wherein the spring is extended to a non-activated position wherein the spring is compressed, and an actuator to permit the spring to be released and drive the plunger rod to move the piston within the container body.

The transfer device of the present invention is typically used to transfer a fluid from one container to another although it more specifically is directed to an arrangement for removing fluid from a first container. There are known devices for accomplishing the same and in this regard, reference may be had to PCT Application CA2008/001125 which discloses such an arrangement and the disclosure of which is hereby incorporated by reference.

The device of the present invention may accomplish the same through the use of a cartridge pack which is an assembly containing the two containers. The transfer device is designed to receive the cartridge pack and then transfer the fluid between the first and second containers.

The outer housing is preferably designed to sit on a base, although it can also be used without such a base. The base preferably presents a sloped upper surface as this assists in the ergonomical operation of the device and to ensure that any air bubbles rise to the top of the container. To achieve the same, the base may have an overall triangular configuration.

The transfer device will also include an outer housing which is designed to receive and retain the cartridge pack. To do so, there may be provided a recessed area having a configuration suitable for holding the cartridge pack. Preferably, the receiving area of the outer housing will include a clasp-like member which is designed to engage with appropriate recesses or apertures in the cartridge pack.

The outer housing is also designed to receive and retain a pivotable lever and to this end, the outer housing may include pin members which would be designed to fit within apertures formed at one end of the lever member.

The lever member, as above mentioned, is pivotally operated and will cause the various actions to take place as will be described in the preferred embodiment hereof. It is understood that the use of the lever is primarily for ergonomic considerations and that other types of arrangements may be utilized. Thus, one could employ an arrangement whereby the different operations are actuated from the rear.

In order to ensure smooth operation of the transfer device, spring members are employed. In a preferred embodiment, two springs are utilized; it suffices to say that one or more can be employed as desired. Thus, one can utilize a larger spring or several smaller springs.

The device preferably utilizes spring guides. The spring guides may range from a shaft about which a spring or springs are placed or alternatively, the guide may extend externally of the springs.

The transfer device also includes an inner housing which is slidable with respect to the outer housing. The inner housing preferably interacts with a plunger rod so as to contact the piston in one container and to provide for an initial displacement thereof. This arrangement is advantageous in that a straight mechanical coupling can be utilized following which the springs are released from their compressed state to continue the motion of the piston.

### BRIEF DESCRIPTION OF THE DRAWINGS

Having thus generally described the invention, reference will be made to the accompanying drawings illustrating an embodiment thereof, in which:

FIG. 1 is a perspective view of the transfer device of the present invention;

FIG. 2 is a top plan view thereof;

FIG. 3 is a side elevational view thereof;

FIG. 4 is an end elevational view thereof;

FIG. 5 is an exploded view illustrating the various components thereof;

FIG. 6 is an exploded side sectional view of the components forming the transfer device; and

FIGS. 6a to 6f illustrate the operation of the transfer device.

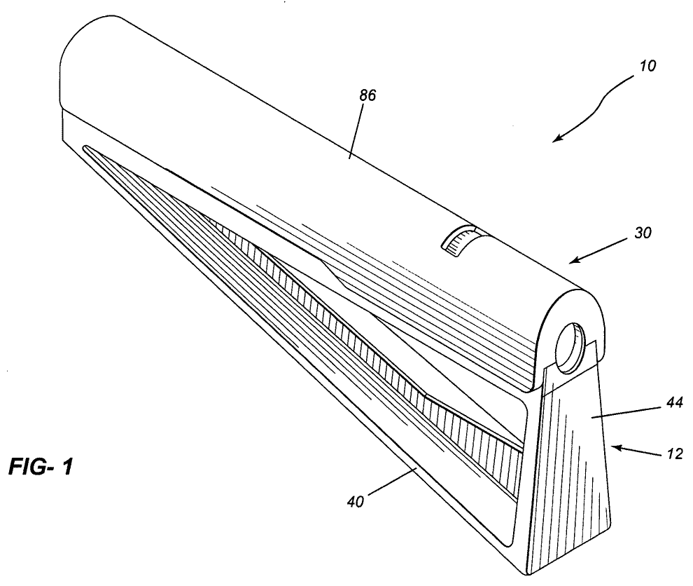

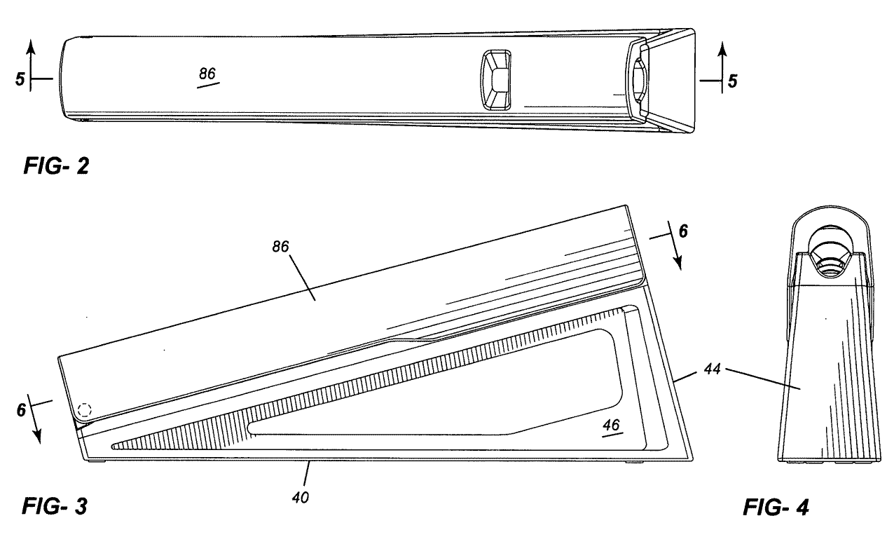

### DETAILED DESCRIPTION OF THE INVENTION

The transfer device of the present invention is designed to be used in the transfer of a fluid from one container to a second container. Ideally, a transfer device of the present invention is utilized in the transfer of a fluid from a first container to a second container such as shown in P.C.T. Application CA2008/001125 and Canadian Application S.N. 2,649,160, filed Jan. 9, 2009, the disclosures of which are hereby incorporated by reference.

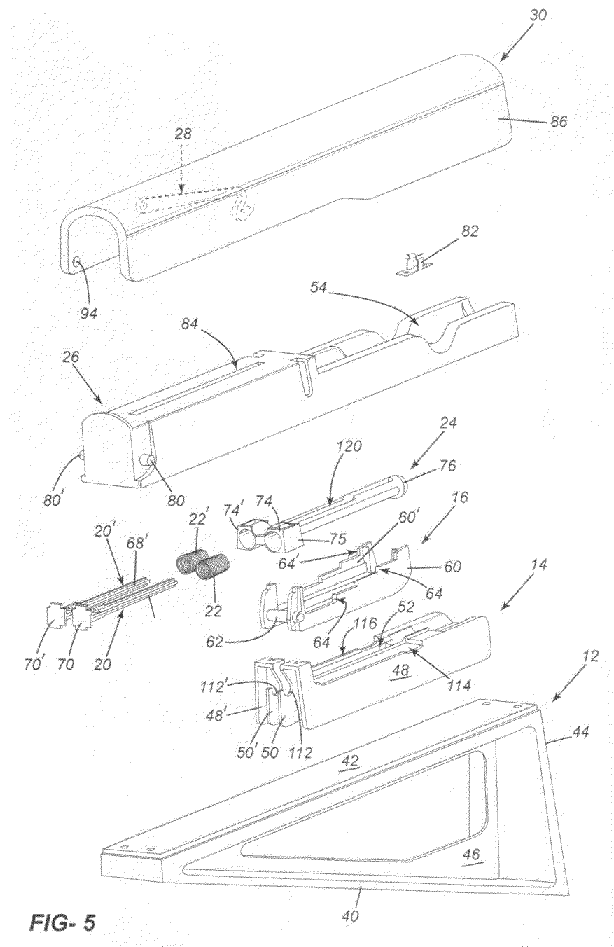

Referring initially to FIG. 5, the transfer device of the present invention is generally designated by reference numeral 10 and includes a base member generally designated by reference numeral 12; an inner housing generally designated by reference numeral 14; a lock member generally designated by reference numeral 16; a stopper generally designated by reference numeral 18; a spring guide generally designated by reference numeral 20; a pair of springs 22, 22′; a plunger rod generally designated by reference numeral 24; an outer housing generally designated by reference numeral 26; an arm generally designated by reference numeral 28; and a lever generally designated by reference numeral 30. A cartridge pack is generally designated by reference numeral 32.

Base member 12, as may be best seen in FIG. 5, includes a bottom wall 40, an upper diagonal wall 42, and an end wall 44. Situated interiorly of the walls is a rib 46.

Inner housing 14, again as best seen in FIG. 5, has a pair of side walls 48, 48′, and a pair of inner walls 50, 50′. Inner walls 50,50′define therebetween a channel 52, for reasons which will become apparent herein below. Channels 114, 116 are formed between walls 48, 50 and 48′, 50′ respectively. Recesses 112, 112′ are formed at the end of inner walls 50, 50′.

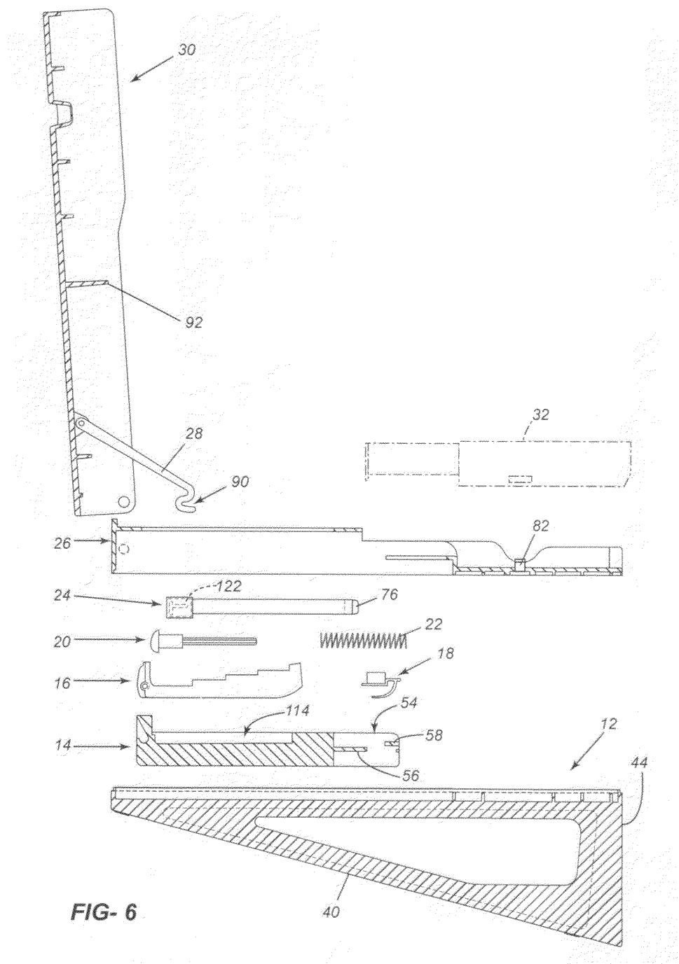

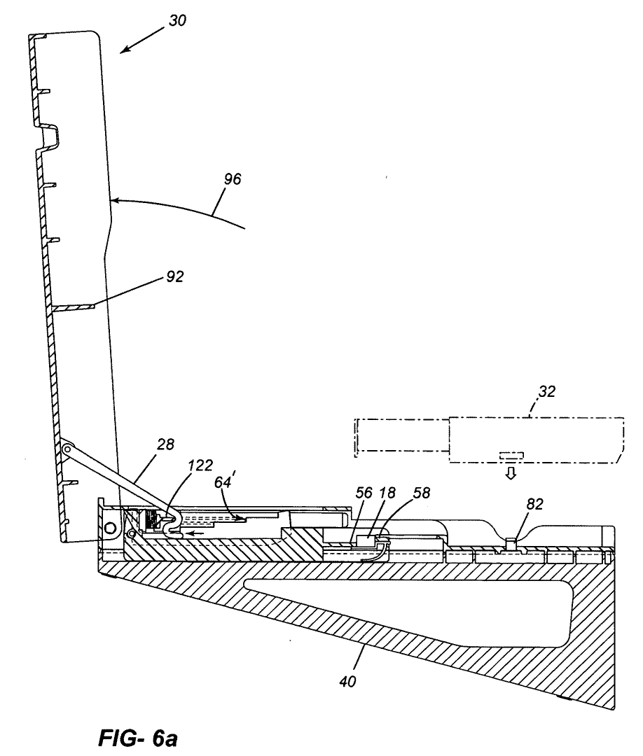

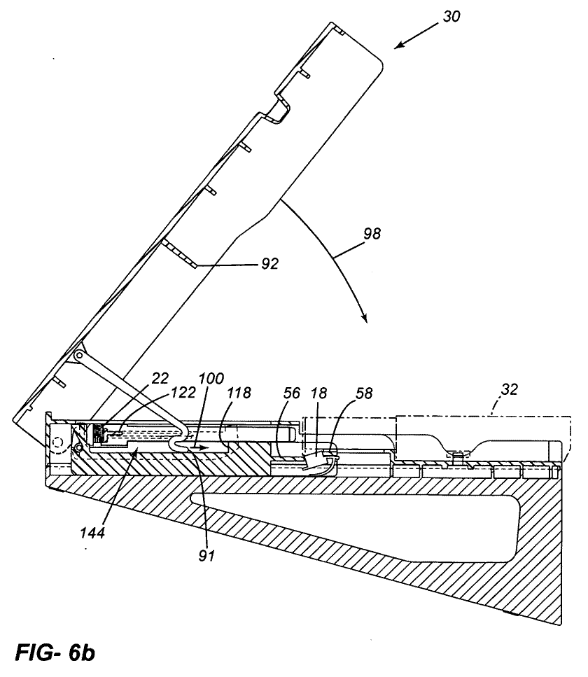

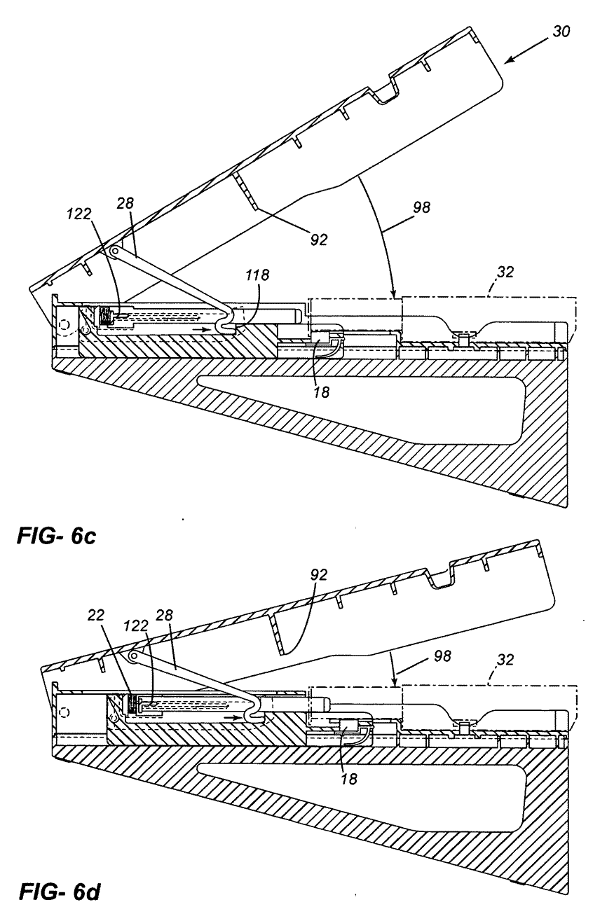

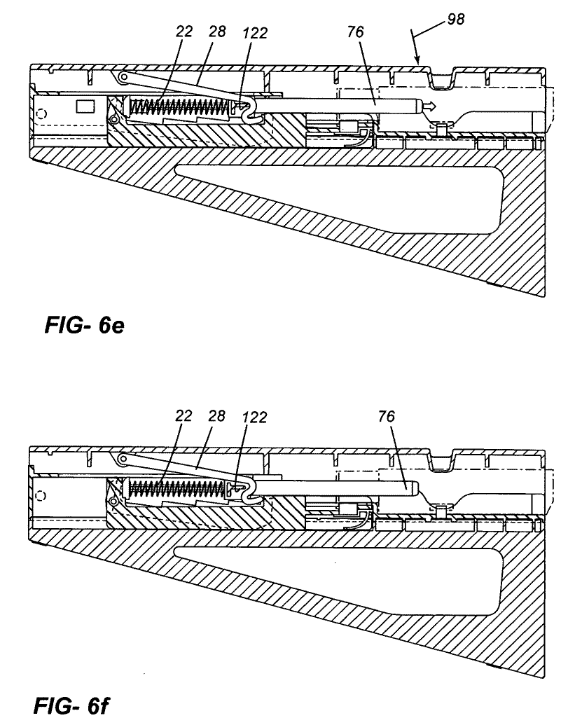

Inner housing 14 also includes a recess 54 (as may best be seen in FIG. 6), having a first flange 56 and a second flange 58 extending inwardly thereof. Stopper 18 initially partially seats within recess 54.

Lock 16 includes side walls 60, 60′ with a pivot pin 62 extending therebetween and which seats in recesses 112, 112′. It will be noted that each of side walls 60, 60′ have a plurality of steps 64, 64′ formed therein.

Spring guides 20, 20′ each comprise a shaft 68, 68′ respectively terminating at a respective end wall 70, 70′.

Plunger rod assembly 24 includes a plunger rod 76 having body portions 75, 75′, and end walls 74, 74′ against which springs 22, 22′ will abut. A channel 120 is formed in plunger rod 76 and a protrusion 122 (FIGS. 6a to 6f) is formed in one of the walls defining channel 120.

Outer housing 26 has a pair of pins 80, 80′ extending outwardly therefrom. Outer housing 26 also includes a metal clasp 82 which is mounted therein for engaging and holding cartridge pack 32. A channel 84 extends longitudinally thereof for reasons described hereinbelow.

Lever 30 has a generally inverted U-shaped body with apertures 94 into which pins 80 are mounted such that lever 30 can pivot with respect to outer housing 26. Arm 28 is pivotably connected to an upper portion of U-shaped body 86; arm 28 has a reverse S-shaped hook portion 90 for reasons which will become apparent hereinbelow. Extending downwardly from U-shaped body 86 is a release actuator 92.

Operation of the device is illustrated in the FIGS. 6a to 6f and reference will now be had thereto.

Initially, cartridge pack 32 is placed on outer housing 26 as illustrated in FIG. 6a. Middle clasp 82 is designed to engage cartridge pack 32; the clasp members may engage an aperture or recess formed within cartridge pack 32.

After it is placed in recess 54, it will be noted that the end of cartridge pack 32 will depress stopper 18 as seen in FIG. 6b. After placement of cartridge pack 32, lever 30 is moved downwardly as indicated by arrow 98 in FIG. 6b. This downward movement will cause hook portion 90 of arm 28 to slide along in a channel 114 as indicated by arrow 100.

Continued movement of lever 30 will further advance hook portion 90 along channel 114 such that it will engage inner housing 46. A slight advancement of inner housing 46 will cause stop member 18 to move upwardly between flanges 56 and 58; this forward motion of inner housing 46 will also allow plunger rod 76 to contact a piston within the vial portion of the cartridge pack and commence movement of the piston.

Continued downward movement of lever 30, as shown in FIGS. 6d and 6e, will result in release actuator 92 contacting an end of lock 16 to cause the same to pivot downwardly. This downward pivoting motion will then free plunger rod 76. Thus, when it is in its upward position, lock 16 engages the inner face of body portion 75 of plunger rod assembly 24 to prevent movement of plunger rod 76. However, with the downward pivoting movement of lock 16, plunger rod 76 is free to advance. This permits springs 22, 22′ to push against end walls 74, 74′ to cause plunger rod 76 to continue exerting force on the piston of the vial of cartridge pack 32.

Forward movement is limited by the engagement of protrusion 122 with hook portion 90 as shown in FIGS. 6e and 6f. Naturally, the arrangement is such that pusher rod 76 has forced a plunger to transfer fluid from a first container to a second container.

In order to prepare the transfer device for further operation, lever 30 is then lifted. Transfer rod assembly 24 will then move rearwardly as a result of the engagement of hook portion 90 with protrusion 122. As a result, lock 16 will again engage body portion 75 to prevent movement of plunger rod 76.

The cartridge pack 32 may then be moved from transfer device 10 and a second cartridge pack inserted. The operation would then be repeated.

It will be understood that the above described embodiments are for purposes of illustration only and that changes and modifications may be made thereto without departing from the spirit and scope of the invention.

I claim:

1. A transfer device for transferring a fluid from a container having a body, an open first end, and a piston within said body, a septum at a second end thereof, the transfer device comprising:
an outer housing, said outer housing having a receiving area to hold and retain a cartridge assembly which includes said container;
a plunger rod aligned with said piston;
a spring positioned to drive said plunger rod in a direction towards said piston;
a retractor to move said plunger rod from an activated position wherein said spring is extended to a non-activated position wherein said spring is compressed; and
an actuator to permit said spring to be released and drive said plunger rod to move said piston within said container body.

2. The transfer device of claim 1 wherein said spring comprises first and second coil springs.

3. The transfer assembly of claim 2 wherein each of said coil springs has a spring guide associated therewith.

4. The transfer device of claim 3 wherein each of said spring guides comprises a shaft located interiorly of a respective coil spring.

5. The transfer device of claim 2 further including a stopper biased to move into a stop position with respect to said plunger rod when said plunger rod is moved into said non-activated position.

6. The transfer device of claim 5 wherein said actuator is designed to contact said stopper to move said stopper away from said plunger to permit said coil spring to move to said extended position by driving said piston.

7. The transfer device of claim 1 further including a clasp in said receiving area to retain a cartridge pack including said container in position.

8. The transfer device of claim 1 further including a pivotable lever attached to said housing, said lever having a member projecting downwardly therefrom, said member acting as said actuator to contact said stopper.

9. The transfer device of claim 8 wherein said pivotable lever includes an arm pivotably connected thereto, said arm having a hook portion at a free end thereof designed to engage said plunger rod to thereby retract said plunger rod when said pivotable lever is moved away from said housing.

10. The transfer device of claim 9 further including an inner housing, said inner housing having a channel formed therein, said inner housing being slidable within said outer housing.

11. The transfer device of claim 10 wherein said free end of said arm is slidable within said channel, said arm being of a length such that upon pivotal movement of said lever towards said outer housing, said hook portion of said of arm will contact a channel end and cause said inner housing to slide with respect to said outer housing and said plunger rod to move and contact said piston to thereby move said piston prior to said actuator permitting said spring to be released.

12. A method for transferring a fluid from a first container to a second container wherein the first container has a body, an open first end and a piston within the body, a septum at a second end thereof, comprising the steps of:
supplying a transfer device having an outer housing, the outer housing having a receiving area to hold and retain a cartridge assembly which includes both said first and second containers, a plunger rod aligned with the piston, spring means positioned to drive said plunger rod in a direction towards said piston, and a retaining means to retain said spring in a compressed position;
moving said plunger rod a distance sufficient to contact and move said piston; and releasing said retaining means to permit said spring means to finish moving said plunger rod.
